(19) United States
(12) Patent Application Publication
(10) Pub. No.: US 2020/0330084 A1
(43) Pub. Date: Oct. 22, 2020

(54) Prostate Exercise Method

(71) Applicant: John Michael Snider, Clinton, TN (US)

(72) Inventor: John Michael Snider, Clinton, TN (US)

(73) Assignee: John M. Snider, Clinton, TN (US)

(21) Appl. No.: 16/386,772

(22) Filed: Apr. 17, 2019

(51) Int. Cl. A61B 17/00; A61M 29/00; A63B 21/00; A63B 23/20; A61H 99/00

(52) U.S. Cl. CPC A61B 17/00; A61M 29/00; A61B 2017/00274; A63B 23/20; A61H 99/00; A63B 21/00185

(57) ABSTRACT

A method for strength and conditioning of the voluntary muscle and its ligaments in the prostate gland. The method uses a device inserted into the urethra to both extend and offer the contracting muscle resistance enabling the exercise of an unopposed muscle. This method reverses the atrophy of this gland known as Benign Prostatic Hyperplasia (BPH) which every man will experience should they live into their nineties.

### CROSS-REFERENCE TO RELATED APPLICATIONS

Provisional Application No. 62/661,828

### TECHNICAL FIELD

A63B “Apparatus for Physical Training, Gymnastics, Swimming, Climbing, or Fencing: Ball Games; Training Equipment,” Specifically: A63B 21/00185 ⋅ “(using resistance provided by the user, e.g. exercising one body part against a resistance provided by another body part).”

### BACKGROUND ART

A61H 1/00 “Apparatus for passive exercising, Vibrating apparatus Chiropractic devices, e.g. body impacting devices, external devices for briefly extending or aligning unbroken bones,” A61M 29/00 “Dilators with or without means for introducing media, e.g. remedies.”

### SUMMARY OF INVENTION

A method for strength and conditioning of the voluntary muscle and its ligaments in the prostate gland. The method uses a device inserted into the urethra to both extend and provide the contracting muscle resistance. This method reverses the atrophy of this gland known as Benign Prostatic Hyperplasia (BPH) which every man will experience should they live into their nineties. Using this method a man can avoid the side effects caused by current treatment methods and attain normal organ function

### Technical Problem

Background and Function/Anatomy

The prostate is a male organ directly below the bladder. A funnel shape portion is around the base of the bladder which directs urine into the urethra within the prostate. Ducts from the testes also pass into the gland. Exiting the gland is the urethra, the tube which passes fluids to the outside of the body via the penis. Both the prostate and the bladder are non rigidly held within the body by ligaments.

The prostate contains two main types of tissue: exocrine glandular tissue and fibro muscular tissue. Exocrine glandular tissue is epithelial* tissue specialized for the secretion of components of semen. *Where the Functions of epithelial cells include secretion, selective absorption, protection, transcellular transport, and sensing Epithelial layers contain no blood vessels, so they must receive nourishment via diffusion of substances from the underlying connective tissue, through the basement membrane.

Prostatic fluid contains enzymes, sugars and alkaline fluids. These aid and stimulate semen mobility by providing a medium for their transport, movement, nutrition and to neutralize acids in the vagina. Much of the prostate is composed of this glandular tissue. Cells of this glandular tissue form small sacs called acini (Latin for berries.) Each acinus (singular) has a duct which carries the fluid from the acinus to larger ducts. The structure can be thought of as something like a cluster of grapes where the grapes have been replaced by a berry form. Some thirty ducts (stems in the grape analogy) combine into two large ducts leading to the urethra. Prostate acinar cells are thought to have a life span of over two years.

Fibro muscular tissue forms the outermost layer of the prostate and tissues surrounding the urethra and is a mixture of smooth muscle and dense irregular connective tissue with many collagen fibers. These muscle and connective structures expel seminal fluid during ejaculation into the urethra and prevent reverse flow into the bladder. At the end of urination these muscles also aid in final bladder emptying.

Various hormones regulate the development of new acinar cells to replace the old ones. As a man passes the age of 40 the healthy prostate can become enlarged due to an excess of prostatic fluid and or acinar cells. This is called BHP—Benign Prostatic Hyperplasia. Half of males age 50 and over are affected and after age 80 ninety (90) percent of males have symptoms. BHP can cause frequent urination, trouble starting to urinate, weak stream, inability to urinate, loss of bladder control and a burning or tingling sensations in the urethra and or testicular ducts. Burning and tingling sensations are likely caused by excess alkaline prostatic fluid leaking into these ducts. Complications can include urinary tract infections, bladder stones and chronic kidney problems.

Previous Treatments

Lifestyle

One can make lifestyle alterations to address the symptoms of BPH include physical activity, decreasing fluid intake before bedtime, moderating the consumption of alcohol and caffeine-containing products and following a timed voiding schedule. Patients can also attempt to avoid products and medications with anticholinerg icproperties that may exacerbate urinary retention symptoms of BPH, including antihistamines, decongestants, opoids, and tricyclic antidepressants; however, changes in medications should be done with input from a medical professional.

Voiding Position

An accommodating voiding position when urinating may influence urodynamic parameters (urinary flow rate, voiding time, and post-void residual volume). A meta-analysis found no differences between the standing and sitting positions for healthy males, but that, for elderly males with lower urinary tract symptoms, voiding in the sitting position:^{[}
decreased the post void residual volume
increased the maximum urinary flow, comparable with pharmacological intervention
decreased the voiding time

This urodynamic profile is associated with a lower risk of urologic complications, such as cystitis and bladder stones.

Medications

Experimentation has found two main medication classes for BPH management, which are alpha blockers and 5α-reductase inhibitors.

Alpha Blockers

Selective α₁-blockers are the most common choice for initial therapy. They include alfuzosin, doxazosin, silodosin, tamsulosin, and terazosin. They have a small to moderate benefit.^{[49]} All five are equally effective but have slightly different side effect profiles.^{[50]} Alpha blockers relax smooth muscle in the prostate and the bladder neck, thus decreasing the blockage of urine flow. Common side effects of alpha blockers include orthostatic hypotension (a head rush or dizzy spell when standing up or stretching), ejaculation changes, erectile dysfunction, headaches, nasal congestion, and weakness.

Tamsulosin and silodosin are selective al receptor blockers that preferentially bind to the α1A receptor in the prostate instead of the alB receptor in the blood vessels. Less-selective α1 receptor blockers such as terazosin and doxazosin may lower blood pressure. The older, less selective al-adrenergic blocker prazosin is not a first line choice for either high blood pressure or prostatic hyperplasia; it is a choice for patients who present with both problems at the same time. The older, broadly non-selective alpha blocker medications such as phenoxybenzamine are not recommended for control of BPH. Non-selective alpha blockers such as terazosin and doxazosin may also require slow dose adjustments as they can lower blood pressure and cause syncope (fainting) if the response to the medication is too strong.

—Reductase Inhibitors

The 5α-reductase inhibitors finasteride and dutasteride may also be used in men with BPH. These medications inhibit the 5α-reductase enzyme, which, in turn, inhibits production of DHT, a hormone responsible for enlarging the prostate. Effects may take longer to appear than alpha blockers, but they persist for many years. When used together with alpha blockers, no benefit was reported in short-term trials, but in a longer term study (3-4 years) there was a greater reduction in BPH progression to acute urinary retention and surgery than with either agent alone, especially in patients were more severe symptoms and larger prostates. Other trials have confirmed reductions in symptoms, within 6 months in one trial, an effect that was maintained after withdrawal of the alpha blocker. Side effects include decreased libido and ejaculatory or erectile dysfunction. The 5α-reductase inhibitors are contraindicated in pregnant women because of their teratogenicity due to interference with fetal testosterone metabolism, and as a precaution, pregnant women should not handle crushed or broken tablets.

Others

Antimuscarinics such as tolterodine may also be used, especially in combination with alpha blockers.^{[62]} They act by decreasing acetylcholine effects on the smooth muscle of the bladder thus helping control symptoms of an overactive bladder.

Phosphodiesterase-5 inhibitors such as sildenafil citrate show some symptomatic relief, suggesting a possible common cause with erectile dysfunction. Tadalafil was considered then rejected by NICE in the UK for the treatment of symptoms associated with BPH. In 2011, the U.S. Food and Drug Administration approved tadalafil to treat the signs and symptoms of benign prostatic hyperplasia, and for the treatment of BPH and erectile dysfunction (ED), when the conditions occur simultaneously.

Self-Catheterization

Intermittent urinary catheterization is used to relieve the bladder in people with urinary retention. Self-catheterization is an option in BPH when it is difficult or impossible to completely empty the bladder. Urinary tract infection is the most common complication of intermittent catheterization. Several techniques and types of catheter are available, including sterile (single-use) and clean (multiple use) catheters, but, based on current information, none is superior to others in reducing the incidence of urinary tract infection.

Surgery

If medical treatment is not effective a person may try office-based therapies or transurethral resection of prostate (TURP), surgery may need to be performed. Surgical techniques used include the following:
Open prostatectomy: not usually performed nowadays, even if results are very good.
Transurethral resection of the prostate (TURP): the gold standard.
Transurethral incision of the prostate (TUIP): rarely performed; the technique is similar to TURP but less definitive.
Photoselective (laser) vaporization of the prostate (PVP): common treatment.

Endovascular

The latest alternative to surgical treatment is arterial embolization, an endovascular procedure performed in interventional radiology. Through catheters, embolic agents are released in the main branches of the prostatic artery, in order to induce a decrease in the size of the prostate gland, thus reducing the urinary symptoms.

Alternative Medicine

While herbal remedies are commonly used, a 2016 review found them to be no better than placebo. Saw palmetto extract from Serenoa repens, while one of the most commonly used, is no better than placebo in both symptom relief and decreasing prostate size. Other herbal medicines include beta-sitosterol from Hypoxis rooperi (African star grass) and pygeum (extracted from the bark of Prunus africana),^{[77]} while there is less substantial support for the efficacy of pumpkin seed (Cucurbita pepo) and stinging nettle (Urtica dioica) root. A systematic review of Chinese herbal medicines found that the quality of studies was insufficient to indicate any superiority over Western medicines.

Also, the ancients found that massaging the prostate trans rectally lessened symptoms. Using the finger, or a cylindrical object such as a drum stick, local pressure can be applied to the gland through the rectal wall. This pressure has reduced symptoms. Before the advent of the medical treatments discussed above, prostate massage was often performed in the medical office by a doctor.

### Solution to Problem

Hypothesis:

BPH correlates with dropping levels of the male hormone testosterone and skeletal muscle atrophy due to age. The atrophied prostate muscles can no longer develop the same pressure within the gland that a young prostate can. Prostatic epithelial cells may continue to live beyond their normal time. The acini may also become over filled with prostate fluid. Thus, the reduced pressure does not cause normal cell expulsion—over populating the gland and or the prostatic fluid may also not be totally emptied enlarging the gland. This is probably why massage does provide some relief. If it were somehow possible to strengthen the muscles of the prostate normal pressures within the gland will occur. Normal pressures create normal acinar cell populations and prostatic fluid volumes. This patent describes a method for strengthening the muscle and ligaments of the prostate restoring the ability to develop normal ejaculatory/urinary pressures within the gland.

### DISCLOSURE

The muscular stroma lies on the front of the prostate and this muscle pulls connective tissue—Facia—reducing the volume of the organ. Muscle contraction expells prostetic fluids and semen into the urethra. The action of this muscle structure can be opposed by inserting a long cylinderical object (a rod) into the meatus of the penis, up through the penile urethra and into the prostate stopping short of the bladder sphincter. Insurtion of the rod into the prostate requires that the penile portion of the urethra be deflected into the same axis as the part of the urethra passing thru the prostate. Using the pubic bone as a fulcrom point the rod can be rotated about the pubic bone displacing the prostatic portion of the urethra, and the glandular portions at the back of the prostate both downward and forward in an arcing path. The muscle of the gland can then be stimulated by the user to return the rod back to the relaxed position against user provided resistance. This is repeated until the muscle has be fully exercised.

To assure safety and comfort the shape of inserted end of the rod and its material of construction are critical. The material of the rod must be sufficiently rigid so as not to deflect under the imposed loads. It also must be sealed so as not allow dirt or bacteria to be accidentally transported into the tissues. And it should be of sufficient length so that the rod can not be lost in the urethra. The shape of the inserted end must both be blunt so as not to poke delicate tissues in the tract but also shaped to gently open both the meatus and enter into the prostate comfortably.

To use the exercise device it must first be cleaned and warmed for both hygiene and comfort. Also an oil or water based lubricant maybe used to reduce friction at the meatus of the penis. A good soap and water wash with a rinse in hot water is all that is required. Olive oil has been found to be comfortable. Selecting the proper size rod for exercise is important.

The french catheter scale is used to measure devices of this type. This scale is based on the diameter of a device in millimeters time 3. Most men are a French 28 which is near three eights of an inch in diameter. With a small amount of lubricant on the opening of the urethra the user inserts the rod while the gentle transitions dialate the meatus slowly. The user must find the most comfortable angle to hold the rod as the insertion progresses. As the rod nears the opening of the prostate it will be roughly forty five degrees from vertical to the front when viewed from the side. Once in the gland insertion should stop before entering the bladder. Some men will hit the bottom of the bladder and feel where to stop insertion. If the rod enters the bladder sphincter it will feel like voiding, and urine will leak if present. Entering the bladder should be avoided due to infection risk and urine leakage. Flexing the muscles used to stop urine flow, which are the prostate muscles, can aid in knowing when the rod is to low within the gland.

With the rod properly positioned exercise may begin. The user relaxes the prostate muscles and moves the free end of the rod upward in an arcing motion without moving it within the prostate. This arcing motion will pivot around the bone of the pelvis. The pelvis is the fulcrom as the rod extends the muscle of the prostate. The user then exerts the prostate muscles against the rod while providing resistance on the other end of the rod. Simultaneously, the user must prevent the expulsion of the device by the action of the gland. He does this by providing a force component axially on the device into the gland. This resistance and contraction should not be enough to cause pain and should allow the muscles to move the rod back to the non-displaced or starting angle of the rod. Like training any other muscle of the body, three to twenty repetitions for three sets every other day should produce good development.

### Advantageous Effects of Invention

This method allows the maintenance and restoration of prostatic function without resorting to treatments which address symptoms without correcting the underlining cause. These treatments have unpleasant side effects which may alter one's lifestyle. The method allows isokinetic, isometric and isotonic exercise of the muscle in the prostate gland through its full range of motion. These types of exercise have been shown to provide development where isometric exercise alone has not shown to be effective against muscle atrophy.

### BRIEF DESCRIPTION OF DRAWINGS

FIG. 1 is a sagittal plane view showing an exercise apparatus (10) inserted into the prostate (70) when the muscle is relaxed. This is also the same position the device attains when the muscle is contracting against the device without any displacing forces. The muscle of the prostate (30) is in the front of the gland and fibrous connective tissue encircles it. Apparatus (10) has displaced the urethra which normally curves upward to the opening (60) of the bladder (50). This defection helps to avoid entering the bladder. Also depicted in FIG. 1 are the anterior section of the pelvis (20) and for reference the prostate half of the seminal duct (40). FIG. 1 represents the beginning and ending point of the exercise repetition.

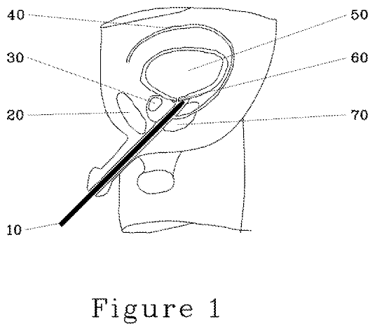

FIG. 2 shows the an exercise apparatus (10) at the extended range of motion of the prostate. The muscle of the prostate (30) is deformed by extension just as is the rest of the gland (70). Soft tissues of the bladder (50) have also distended accommodating the movement of the prostate. Similarly, the bladder opening (60) has moved in an arcing motion prescribed by the device (10). The user then provides a motion and resistive force profile with his hands to achieve maximum muscle structure stimulation.

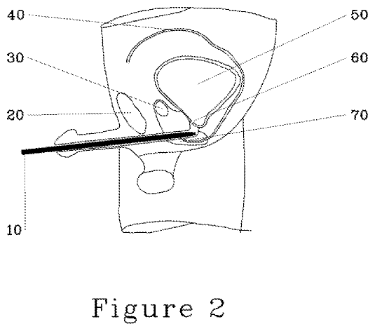

A great number of variations of this concept are possible. The rod could be curved, or have an elliptical or other crossectional shape, made of multiple materials, painted wood, carbon fiber or any appropriate plastic—both reinforced or not. In light of this I make the following Claims:

### Patent Literature

U.S. Pat. No. 8,182,503
0017

### Non Patent Literature

1. Tunn, Sabine & Nass, Ralf & Ekkernkamp, A & Schulze, Harald & Krieg, Michael. (1989). Evaluation of average life span of epithelial and stromal cells of human prostate by superoxide dismutase activity. The Prostate. 15. 263-71. 10.1002/pros.2990150307.
2. 26331999 Kim, E H; Larson, J A; Andriole, G L (2016). “Management of Benign Prostatic Hyperplasia”. Annual Review of Medicine (Review). 67: 137-51. doi:10.1146/annurev-med-063014-123902. PMID.
3. Wikipedia, https://en.wNikipedia.orgIwiki/Benign_prostatic_hyperplasia, see Management Section.
4. https:/www.nasa.gov/vision/earth/livingthings/10dec_muscles html

1. A method for exercising the muscle of the prostate comprising: inserting into the urethral meatus an object of suitable dimensions and rigidity through the urethra and into the portion of the urethra that is surrounded by the prostate without entering the bladder; extending the muscle of the prostate by using the foregoing object as a lever where the fulcrum is made about the pelvis; strengthening the prostate muscle by contracting it against the objects counter acting force supplied by the user; engaging an exercise regiment that increases strength, flexibility and range of motion of the prostate muscle and ligament structure; restoring prostate internal pressures so that fully matured end of life acinar cells and or any excess prostatic fluid are expelled allowing the organ to become healthy again.

2. The method in claim 1 further comprising the use of an object composed of a nonporous, smooth and rigid material that provides the exercise forces and is both hypoallergenic and easily cleaned.

3. The method in claim 1 further comprising the use of an object described in claim 2 shaped on each end so as to provide easy passage into the urethra and prostate so as to minimize user discomfort.

4. The method in claim 1 further comprising the use of an object which is long enough so as not to get lost in the urethra and to give the user a place to apply prostate muscle counter acting force.

5. The method in claim 1 further comprising utilizing the use of an object described in claims 2 thru 4 to stretch the muscles and ligaments of the prostate before or after strengthening exercises to stimulate the muscle before conditioning exercises and after exercises to gain and or avoid loss or range of motion.

6. The method in claim 1 further comprising the control of the location of the exercise device within the prostate by the user as the exercise proceeds through the range of motion.

7. The method in claim 1 further comprising utilizing the object described in claims 2 thru 4 to exercise the muscles and ligaments of the prostate wherein the number of repetitions per set, the number of sets per exercise session and the recovery interval between sessions is chosen to maximize prostate muscle performance.

8. The method in claim 1 further comprising utilizing the object described in claims 2 thru 4 wherein the insertion step is aided by use of a water or oil based lubricant.
